(19) United States
(12) Patent Application Publication
(10) Pub. No.: US 2020/0339962 A1
(43) Pub. Date: Oct. 29, 2020

(54) PROTEUS MIRABILIS PHAGE RDP-SA-16033 AND INDUSTRIAL PRODUCTION PROCESS THEREOF

(71) Applicant: Recom (Qingdao) biotechnology Co., Ltd., Qingdao (CN)

(72) Inventors: Xiansheng Li, Jinan (CN); Xinyong Du, Jinan (CN); Yuqing Liu, Jinan (CN); Qing Zhang, Jinan (CN); Chengsheng Luo, Jinan (CN); Ruxia Ma, Jinan (CN); Dandan Zhao, Jinan (CN)

(21) Appl. No.: 16/926,799

(22) Filed: Jul. 13, 2020

(30) Foreign Application Priority Data
Nov. 12, 2019 (CN) 201911098168.X

(51) Int. Cl. C12N 7/00

(52) U.S. Cl. CPC C12N 7/00; C12N 2795/00051

(57) ABSTRACT

The present invention discloses a Proteus mirabilis phage RDP-SA-16033. The host of the Proteus mirabilis phage is Proteus mirabilis S5. The phage may form a plaque having a diameter of 4-6 mm on a double-layer plate. When observed through an electron microscope, the phage has a head in polyhedral cubic symmetry, is coated by nucleic acid, has a diameter of about 70 nm, has a tail of about 150 nm long, has a tail sheath, has a neck connected to the head and the tail, and belongs to tailed virales myovirus division. The present invention further provides a production process of the phage. After centrifugation of value-added liquid, a titer of the phage is increased by membrane concentration, and then residual host and other infectious microbes in value-added are effectively removed by a ceramic membrane and a 0.22 μm polyethersulfone filter membrane. Meanwhile, the phage is reserved to the utmost extent.

### TECHNICAL FIELD

The present invention relates to the field of microbes, and particularly relates to a Proteus mirabilis phage RDP-SA-16033 and an industrial production process thereof.

### BACKGROUND

Proteus mirabilis is a Gram-negative bacterium, has no spore or capsule, is full of flagella on the whole body, has a form in apparent pleomorphism, is low in nutritional requirements during growth and reproduction, and may reproduce at 4-7° C. The Proteus mirabilis widely exists in human and animal wastes, sewage, soil and clinical specimens, may cause poisoning due to produced toxins, and is a common conditioned pathogen. The Proteus mirabilis may infect common domestic animals such as chickens, pigeons, goats, dairy cattle and pigs, may also infect other animals such as monkeys, foxes, pandas and minks, and can cause surgical infection, urinary system infection, diarrhea, bacteremia and the like when body resistances of the animals is decreased. In recent years, in places such as Henan, Hebei, Shandong, Anhui and Guangxi in China, continuous outbreaks of Proteus mirabilis diseases occur in chicken flocks, particularly during seasonal change, environmental change, population transfer and mixed infection of other pathogens, resistance of the chicken flocks is decreased, the condition of the chickens is worsened, a case fatality rate is increased, and great economic losses are brought to livestock breeding.

In addition, with gradual increase of bacterial resistance, the bacterial resistance problem cannot be solved by original antibiotic therapy. Prevailing strains may be locked by epidemiology of pathogenic bacteria, and when phages are screened from the prevailing strains, therapeutic effects of the phages will increasingly show incomparable superiority to antibiotics. With the gradual improvement and application of molecular biology, a lysis spectrum of the phage is expected to be further extended, the lysis capability of the phage may be enhanced, and phage therapy in infectious diseases caused by the pathogenic bacteria will also show an unprecedented development space.

The present invention aims to solve the problems of the bacterial resistance and antibiotics abuse in response to the national order of comprehensive resistance forbidding, and provide a safe and reliable lytic Proteus mirabilis phage capable of treating the Proteus mirabilis diseases of livestock.

### SUMMARY

One of the purposes of the present invention is to provide a safe and reliable lytic Proteus mirabilis phage RDP-SA-16033 capable of treating Proteus mirabilis diseases of livestock, and provide a pathogenic bacterium S5 for producing the phage. Meanwhile, the present invention provides an efficient method for separating the phage.

Another purpose of the present invention is to provide applications of the phage in treatment of the Proteus mirabilis diseases of livestock, and provide applications of the pathogenic bacterium in producing the phage in livestock breeding. The RDP-SA-16033 has high thermal tolerance and wide pH tolerance range, and is high in titer, safe, effective and favorable for industrial production. In combination with a value-added characteristic of the phage, a large-scale production method is provided.

To realize the above purposes, the present invention provides the following technical solutions:

A Proteus mirabilis phage RDP-SA-16033 is provided, and a host of the Proteus mirabilis phage RDP-SA-16033 is Proteus mirabilis S5. The phage may form a plaque having a diameter of 4-6 mm on a double-layer plate. When observed through an electron microscope, the phage has a head in polyhedral cubic symmetry, is coated by nucleic acid, has a diameter of about 70 nm, has a tail of about 150 nm long, has a tail sheath, has a neck connected to the head and the tail, and belongs to tailed virales myovirus division. A collection number of the phage is CGMCC No. 18197.

An industrial production method of the Proteus mirabilis phage RDP-SA-16033 includes the following steps:

(1) seed preparation, including host seed preparation and phage seed preparation, wherein,

A, the host seed preparation: inoculating a single colony to a 5 mL of LB fluid medium in a sterile environment, and performing culture at 37° C. and 200 rpm for 4-6 h; inoculating the cultured host bacteria to a 100 mL of LB fluid medium at an inoculum size of 5%, and performing culture at 37° C. and 200 rpm for 4-6 h; inoculating the host bacteria to a 1000 mL of LB fluid medium at an inoculum size of 5%, and performing culture at 37° C. and 150 rpm for 4-6 h; and placing the cultured host bacteria seeds in a refrigerator at 4° C. for later use;

B, the phage seed preparation: properly diluting preserved phage seeds; placing 200 μL of the phage seeds and 200 μL of the host bacteria in an upper medium; pouring the medium into a double-layer plate; performing culture at 37° C. for 12-16 h; picking a plaque under a sterile condition, and placing the plaque in 1 mL of normal saline at 12000 rpm for 10 min; respectively inoculating the liquor and the host to a 5 mL of LB medium according to a ratio of 2%, and performing culture at 37° C. and 200 rpm for 4-8 h; simultaneously inoculating the cultured phage and the host to a 100 mL of LB fluid medium at an inoculum size of 2°/o, and performing culture at 37° C. and 200 rpm for 4-8 h; simultaneously inoculating the cultured phage and the host to a 1000 mL of LB fluid medium at an inoculum size of 2%, and performing culture at 37° C. and 200 rpm for 4-8 h; and placing the cultured phage seeds in the refrigerator at 4° C. for later use;

(2) phage proliferation: inoculating 3% of the host bacteria in a seed tank; after culturing for 4-6 h, controlling OD600 to 0.6-0.8; inoculating the phage seeds at an inoculum size of 3%, and performing culture for 6-8 h until dissolved oxygen bubbles and the pH tends to be stable;

inoculating the prepared host bacteria in a seed fermenter according to a ratio of 3%; after culturing for 3-4 h, controlling the OD600 value to 0.6-0.8; inoculating the host bacteria in the fermenter by virtue of a seed transfer tube at an inoculum size of 5%; culturing the host in the seed tank for 4-6 h; inoculating the phage at an inoculum size of 5% when the OD value is 0.6-0.8, and performing culture for 6-8 h; and ending proliferation when the dissolved oxygen bubbles and the pH tends to be stable;

(3) phage after-treatment process:

A, centrifugation: centrifuging the completely proliferated phage by a tube centrifuge at a rate of 14000 rpm and flow velocity of less than or equal to 30 L/H, and removing non-lysed host and bacteria debris;

B, performing concentration by a spiral-wound membrane so as to increase a titer of the phage, wherein a specification of the spiral-wound membrane is 50 kd;

C, performing filtration by a 500 nm ceramic membrane, and removing partial bacteria debris and residual host;

D, filtration sterilization: adopting three-stage filtration: sterilizing by 0.45 μm polypropylene, 0.2 μm double-layer polypropylene and 0.22 pin polyether sulfone;

(4) low-temperature spray drying of the phage:

adding a carrier into the filtered and sterilized phage liquid, and uniformly stirring; performing low-temperature spray drying at a drying temperature of 60° C. and a feed rate of 8 L/H, thereby obtaining phage powder;

Preferably, in the step (2), parameters of the seed tank are as follows: 200-300 rpm, 37° C., ventilating 1: (0.6-0.8) vvm; parameters of the fermenter are as follows: 120-150 rpm, 37° C., ventilating 1: (0.6-0.8) vvm.

Preferably, in the step (2), the medium includes the following components: 2% of yeast extract powder, 0.5% of glycerin, 0.5% of sodium chloride, 0.1% of monopotassium phosphate, 0.1% of dipotassium phosphate, 20 ppm of calcium chloride, 20 ppm of magnesium chloride and the balance of water.

Preferably, in the step (4), the carrier includes the following components in percentage by mass: 3% of soluble starch, 3% of polyvinylpyrrolidone, 3% of lactose, 2% of trehalose, 0.2% of disodium hydrogen phosphate, 0.3% of sodium dihydrogen phosphate, 0.1% of vitamin C and 0.5% of modified chitosan.

The present invention has beneficial effects as follows:

(1) In the present invention, the Proteus mirabilis pathogenic bacterium S5 is discovered and separated, and the Proteus mirabilis phage RDP-SA-16033 is prepared through separation by taking the Proteus mirabilis pathogenic bacterium S5 as the host. The phage RDP-SA-16033 has strong lysis effectiveness on the Proteus mirabilis pathogenic bacterium S5 in a breeding environment, and provides a phage source for the phage in industrial production in prevention and treatment of the Proteus mirabilis pathogenic bacterium S5 in the breeding environment.

(2) The RDP-SA-16033 has high thermal tolerance and wide pH tolerance range, and is high in titer, safe and effective.

(3) The present invention provides a method favorable for large-scale industrial production, the process cost is relatively low, and the prepared product is excellent in stability and convenient for popularization and application.

### DESCRIPTION OF DRAWINGS

The above and other features, properties and advantages of the present invention become more apparent through the description of drawings and embodiments below.

FIG. 1 is a schematic diagram of a plaque separated in the present invention by virtue of electron microscopy observation.

FIG. 2 is a schematic diagram of a one-step growth curve experiment of a phage in the present invention.

FIG. 3 is a schematic diagram of a pH stability experiment of a phage in the present invention.

FIG. 4 is a schematic diagram of a lysis experiment of a phage in the present invention to a host.

FIG. 5 is a schematic diagram of a heat stability experiment of a phage in the present invention.

FIG. 6 is a schematic diagram of a genetic stability experiment of a phage in the present invention.

### DETAILED DESCRIPTION

The present invention is further described below in combination with drawings and embodiments. The following embodiments are favorable for those skilled in the art to further understand the present invention, but not limit the present invention in any form. It shall be indicated that, several variations and improvements may be further made by those ordinary skilled in the art without deviating from the concept of the present invention. These variations and improvements belong to the protection scope of the present invention.

### Embodiment 1 Separation and Identification of Pathogenic Proteus mirabilis S5

Sampling was performed from a diseased farm; livers of diseased livestock were taken by virtue of sterile operations; a selective medium was scribed; after culture at 37° C. for 18-24 h, a round and flat red colony with neat edges and a smooth and wet surface was formed on the medium; a typical colony was selected to be continuously scribed and purified for 3 times; then a single colony was selected and inoculated to a 5 mL of LB broth medium, and shaking culture was performed at 37° C. and 200 rpm for 8 h so as to obtain uniform and turbid bacterial suspension; the bacterial suspension is determined as the pathogenic Proteus mirabilis by virtue of 16sRNA molecular identification and serotype identification, wherein one strain was named as S5; and the pathogenic Proteus mirabilis is preserved in a refrigerator at minus 80° C. by adopting a glycerin preservation method.

### Embodiment 2 Separation and Identification of Phage RDP-SA-16033

Sample treatment: epidemic materials were collected from a broiler farm; livers were taken out after dissection; the livers were ground by a sterilized grinder; then normal saline in an amount of 2 times of the volume was added; oscillation was performed for 10 min; centrifugation was performed at 12000 rpm for 10 min; the supernatant was taken to pass through a 0.22 μm filter; and the filtrate was collected for later use.

Concentration of the phage: 10% of GEG8000 was added into the collected liquor, standing was performed at 4° C. for 4-6 h; centrifugation was performed at 12000 rpm for 10 min; the liquor was removed; a precipitate was dissolved with a small amount of normal saline; and dissolved liquid was collected for later use.

Proliferation of the phage: 0.2 mL of bacterial suspension and 0.5 mL of the dissolved liquid were added into 5 mL of LB broth; shaking culture was performed at 37° C. and 200 rpm overnight; centrifugation was performed at 12000 rpm for 5 min; the supernatant was taken to pass through a 0.22 μm filter; and the filtrate was collected for later use.

Phage separation: phage separation was performed by adopting a double plate method; after 0.5 mL of mixed bacterial suspension filtrate and 0.2 mL of Proteus mirabilis bacterial suspension were uniformly mixed, the mixture was placed in a water bath at 37° C. for 10 min; a double plate was laid, the mixture was cultured in an incubator at 37° C. for 6-8 h, and results were observed. If the phage existed, a transparent plaque existed on the plate. The transparent plaque was collected to be added into 1 mL of normal saline; the liquid was placed in a water bath at 37° C. for 30 min; 0.1 mL of leach liquor and 0.2 mL of bacterial suspension were placed in the water bath at 37° C. for 10 min; the double plate was laid, the liquid was cultured in an incubator at 37° C. for 6-8 h for purification; purification was performed for 2-3 times according to the above step until a size of the plaque was uniform, thereby obtaining the phage, wherein the diameter of the plaque is 4 mm-6 mm, and the phage was named as RDP-SA-16033.

Collection of the phage: single plaques were picked up, added into 2 mL of normal saline, and placed in a water bath at 37° C. for 10 min; after appropriate gradient dilution, 0.1 mL of diluent and host were respectively uniformly mixed; the double plate was laid; a plate on which the plaque is cloudlike was selected; an upper layer of the double-layer plate was cleaned with 5 mL of sterilized LB broth medium and then collected into a centrifuge tube; centrifugation was performed at 12000 rpm for 10 min; the liquor is enabled to pass through a 0.22 μm filter membrane; the filtrate was collected; then 2% of chloroform was added and placed at a room temperature for 12-24 h; 1 mL of the filtrate was added into an ampoule bottle; the opening was sealed by an alcohol blast burner; marking was performed; and the product was collected in a refrigerator at 4° C. for a long time.

The prepared phage was subjected to gene sequencing. The phage has a gene length of 41327 bp, and has no virulence gene or lysogenic gene.

### Embodiment 3: Electron Microscopy Observation of Phage

20 μL of liquid containing coarse phage particles was dropped onto a copper screen; natural precipitation was performed for 15 min; excessive liquid was sucked away by the side face with filter paper; a drop of 2% phosphotungstic acid (PTA) was added onto the copper screen for staining the phage for 10 min; staining fluid was sucked away from the side face with the filter paper; and after the sample was dried, morphology of the phage was observed by an electron microscope.

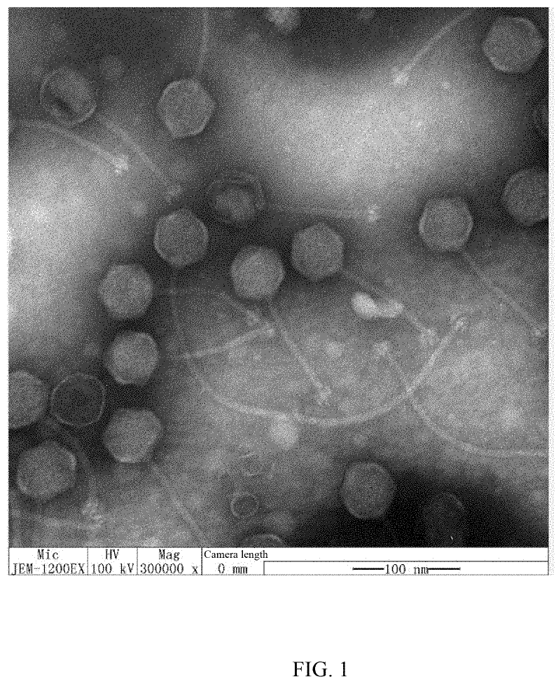

The phage RDP-SA-16033 has a head in polyhedral cubic symmetry, is coated by nucleic acid, has a diameter of about 70 nm, has a tail of about 150 nm long, has a tail sheath, and has a neck connected to the head and the tail. According to the ninth report of virus classification organized by international virus taxology, the phage may be classified as tailed virales myovirus division, referring to FIG. 1.

### Embodiment 4: An Industrial Production Method of the Proteus mirabilis Phage RDP-SA-16033 Includes the Following Steps

1. Seed Preparation

(1) Host seed preparation: a single colony was inoculated to a 5 mL of LB fluid medium in a sterile environment, and cultured at 37° C. and 200 rpm for 4-6 h; the cultured host bacteria were inoculated to a 100 mL of LB fluid medium at an inoculum size of 5%, and cultured at 37° C. and 200 rpm for 4-6 h; the host bacteria were inoculated to a 1000 mL of LB fluid medium at an inoculum size of 5%, and cultured at 37° C. and 150 rpm for 4-6 h; and the cultured host bacteria seeds were placed in a refrigerator at 4° C. for later use.

(2) The phage seed preparation: preserved phage seeds were properly diluted; 200 μL of the phage seeds and 200 μL of the host bacteria were placed in an upper medium; the medium was poured into a double-layer plate; culture was performed at 37° C. for 12-16 h; a plaque was picked under a sterile condition, and placed in 1 mL of normal saline at 12000 rpm for 10 min; the liquor and the host were respectively inoculated to a 5 mL of LB medium according to a ratio of 2%, and cultured at 37° C. and 200 rpm for 4-8 h (cultured until proliferation liquid was clarified or floccules appeared); the cultured phage and the host were simultaneously inoculated to a 100 mL of LB fluid medium at an inoculum size of 2%, and cultured at 37° C. and 200 rpm for 4-8 h; the cultured phage and the host were simultaneously inoculated to a 1000 mL of LB fluid medium at an inoculum size of 2%, and cultured at 37° C. and 200 rpm for 4-8 h; and the cultured phage seeds were placed in the refrigerator at 4° C. for later use.

2. Phage Proliferation:

3% of the host bacteria were inoculated in a seed tank; after culture for 4-6 h, OD600 was controlled to 0.6-0.8; the phage seeds were inoculated at an inoculum size of 3%, and cultured for 6-8 h until dissolved oxygen bubbles and the pH tends to be stable.

The prepared host bacteria were inoculated in a seed fermenter according to a ratio of 3%; after culture for 3-4 h, the OD600 value was controlled to 0.6-0.8; the host bacteria were inoculated in the fermenter by virtue of a seed transfer tube at an inoculum size of 5%; the host was cultured in the seed tank for 4-6 h; the phage was inoculated at an inoculum size of 5% when the OD value is 0.6-0.8, and cultured for 6-8 h; and proliferation was ended when the dissolved oxygen bubbles and the pH tends to be stable.

Parameters of the seed tank are as follows: 200-300 rpm, 37° C., ventilating 1: (0.6-0.8) vvm; parameters of the fermenter are as follows: 120-150 rpm, 37° C., ventilating 1: (0.6-0.8) vvm.

The medium includes the following components: 2% of yeast extract powder, 0.5% of glycerin, 0.5% of sodium chloride, 0.1% of monopotassium phosphate, 0.1% of dipotassium phosphate, 20 ppm of calcium chloride, 20 ppm of magnesium chloride and the balance of water.

3. Phage after-Treatment Process

(1) Centrifugation: the completely proliferated phage was centrifuged by a tube centrifuge at a rate of 14000 rpm and flow velocity of less than or equal to 30 L/H, and non-lysed host and bacteria debris were removed;

(2) concentration was performed by a spiral-wound membrane so as to increase a titer of the phage, wherein a specification of the spiral-wound membrane was 50 kd;

(3) filtration was performed by a 500 nm ceramic membrane, and partial bacteria debris and residual host were removed; and

(4) filtration sterilization: three-stage filtration was adopted (0.45 μm polypropylene, 0.2 μm double-layer polypropylene and 0.22 μm polyether sulfone) for sterilization.

4. Low-Temperature Spray Drying of the Phage

A carrier was added into the filtered and sterilized phage liquid, and uniformly stirred, wherein the carrier includes the following components in percentage by mass: 3% of soluble starch, 3% of polyvinylpyrrolidone, 3% of lactose, 2% of trehalose, 0.2% of disodium hydrogen phosphate, 0.3% of sodium dihydrogen phosphate, 0.1% of vitamin C and 0.5% of modified chitosan; and low-temperature spray drying was performed at a drying temperature of 60° C. and a feed rate of 8 L/H, thereby obtaining phage powder.

Under conditions of 37° C. and 50° C., the phage powder subjected to low-temperature spray drying and vacuum freeze drying in the present invention has stability as follows:

Under the condition of 37° C., after the phage powder obtained by the process in the present invention is stored for 180 days, the titer of the phage is decreased from initial 11.96 Log(pfu/g) to 10.31 Log(pfu/g), while a titer of the phage powder adopting vacuum freeze drying is decreased from initial 11.63 Log(pfu/g) to 8.16 Log(pfu/g). Under the condition of 37° C., the phage powder produced by low-temperature spray drying has excellent stability.

Heat stability of the two kinds of phage powder is further compared. A storage temperature is raised to 50° C. At 50° C., within 120 min, the titer of the phage powder produced by low-temperature spray drying is decreased from initial 11.96 Log(pfu/g) to 8.61 Log(pfu/g), while the titer of the phage powder adopting vacuum freeze drying is decreased from initial 11.63 Log(pfu/g) to 7.31 Log(pfu/g). Similarly under the condition of 50° C., the phage powder produced by low-temperature spray drying in the present invention has excellent stability.

### Embodiment 5 Determination of Phage Growth Curve

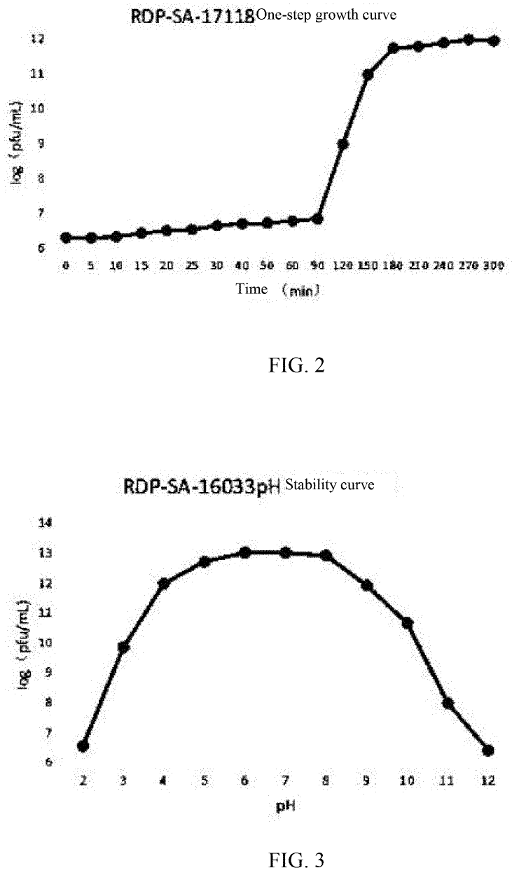

Proteus mirabilis suspension in a logarithmic phase was taken; Proteus mirabilis S5 and a phage RDP-SA-16033 were inoculated according to a ratio of optimal multiplicity of infection, placed in a water bath at 37° C. for 10 min, and centrifuged at a room temperature at 12000 r/min for 5 min; the supernatant was removed; free phages that were not adsorbed onto the host were removed; the liquor was washed with an LB broth medium twice; a precipitate was re-suspended in the LB broth medium at 37° C., and rapidly placed in a shaker at 200 r/min at 37° C. for performing shaking culture, and timing was performed; sampling counting was performed within previous 30 min every 5 min, counting was performed within 30-60 min every 20 min, and counting was performed within 60-300 min every 30 min; time was taken as horizontal coordinate, phage titer log value was taken as a vertical coordinate, a growth curve was drawn to obtain an incubation period and a lysis period of the phage, and the mean lysis amount was calculated. The mean lysis amount is equal to phage titer at the end of outbreak/concentration of host bacteria during early infection. The results are shown in FIG. 2.

Referring to FIG. 2, the titer of the phage infected with the host bacteria has no obvious change within 90 min, which indicates that the incubation period of the phage is about 90 min. Within 90-180 min after infection, the titer of the phage is obviously increased and then tends to be stable, which indicates that the lysis period of the phage is about 90 min. Through calculation, the lysis amount of the phage is about 260 PFU/infected cells, which indicates that the phage has extremely high lysis and replication abilities.

### Embodiment 6 Determination of pH Stability of the Phage

A pH value of normal saline was regulated with diluted hydrochloric acid and a NaOH solution; buffer solutions having pH values such as 2, 3, 4, 5, 6, 7, 8, 9, 10, 11 and 12 were prepared; the phage was diluted into 1*10 10 pfu/mL with the prepared buffer solutions; the diluent was diluted with the normal saline in a water bath at 37° C. for 1 h by 10 times, and a double plate was laid; inverted culture was performed at 37° C. for 4-6 h, and counting was performed. It can be seen from the table, the optimum growth pH value of the RDP-SA-16033 is a neutral environment. The results are shown in FIG. 3.

After cultured under different pH conditions for 1 h, the phage RDP-SA-16033 may maintain excellent activity in a pH range of 5.0-8.0, and the phage titer has no obvious change. Moreover, at the pH value of 7.0, the phage titer is the highest. With increase or decrease of the pH value, the titer of the phage is obviously decreased, which indicates that the phage RDP-SA-16033 may tolerate weak acid and weak base conditions, and has excellent pH tolerance.

### Embodiment 7 Determination of Optimal Multiplicity of Infection

According to a ratio of the optimal multiplicity of infection such as 100, 10, 1, 0.1, 0.01, 0.001 and 0.0001, phage proliferation fluid and a host were added into LB broth, and the total volume of a culture system was ensured to be the same; after shaking culture at 37° C. and 200 rpm for 8 h, centrifugation was performed at a room temperature at a rate of 12000 r/min for 5 min; and the supernatant was spread on a double plate, and the titer of the phage was determined. The results are shown in Table 1.

TABLE 1
Determination Results of Optimal Multiplicity of Infection

| | Number of | Number of | | |
|---|---|---|---|---|
| | proteus | phages RDP- | | 8 h phage |
| Tube | mirabilis | SA-16033 | Multiplicity | titer |
| No. | S5 (cfu/mL) | (pfu/mL) | of infection | (pfu/mL) |
| 1 | 1*10⁸ | 1*10¹⁰ | 100 | 1.83*10¹² |
| 2 | 1*10⁸ | 1*10⁹ | 10 | 2.51*10¹² |
| 3 | 1*10⁹ | 1*10⁹ | 1 | 6.03*10¹² |
| 4 | 1*10⁹ | 1*10⁸ | 0.1 | 7.23*10¹² |
| 5 | 1*10⁹ | 1*10⁷ | 0.01 | 9.86*10¹² |
| 6 | 1*10⁹ | 1*10⁶ | 0.001 | 4.23*10¹² |
| 7 | 1*10⁹ | 1*10⁵ | 0.0001 | 1.06*10¹² |

The results show that, when the multiplicity of infection is 0.01, after culture of 8 h, the proliferation fluid is relatively clear, and the titer is the highest, which indicates that the optimal multiplicity of infection of the RDP-SA-16033 is 0.01.

### Embodiment 8 Determination of Lysis Curve of Phage to Host

The phage was added into bacterial suspension, and due to lysis of the phage to the host, the OD600 value was changed, so that a lysis curve of the phage to the host was obtained. Host bacteria suspension cultured for 6 h was inoculated to a 100 mL of LB medium according to a ratio of 1:100; phage proliferation fluid was added into 3 tubes according to the optimal multiplicity of infection; and the host without the phage was taken as control, and the mean value was taken as the result. The result is as shown in FIG. 4.

Referring to FIG. 4, for the host without the phage, with the proliferation of the host, the OD600 value is gradually increased and achieves a balance within 330 min; while for the host added with the phage, within the previous 20 min, the phage is located at a stage of adsorbing and infecting the host, and the OD600 value is increased; with index increase of the quantity of the phages, within 40 min, the host is lysed by the phage, the OD600 value is not obviously increased, but with the extension of time, the OD600 value is gradually increased.

When the Proteus mirabilis RDP-SA-16033 is added into host bacteria S5 suspension, a certain lysis effect is achieved within 40 minutes after action. Compared with the control group, for the host without the phage, the OD600 value is obviously increased, which indicates that the phage has high lysis ability to the host and may continuously lyse the host bacteria. Since the phage is located at the stage of adsorbing and infecting the host and only a small part of phages start to lyse the host within the previous 20 minutes, the amount of lysing the host is far less than the amount of increase of the host. Therefore, the OD600 value still maintains a certain rising trend within the previous 20 minutes. With the index increase of the quantity of the phages, the quantity of lysed host is more than a proliferation rate of the host within 40 minutes, so the OD600 value starts to gradually decrease, and the mixed bacteria solution is the most clarified. Within 270 min after continuous action, the mixed bacteria solution becomes turbid again, and original sensitive bacteria are lysed by the phages and alternate with a flora substituted by mutant tolerance bacteria. Therefore, to obtain high-titer phages, the mixed bacteria solution should be controlled in a time range from a moment of reaching the highest clarity to a moment before a turbidity return phenomenon starts, thereby avoiding an influence on the titer of the phage.

### Embodiment 9 Determination of Heat Stability of Phage

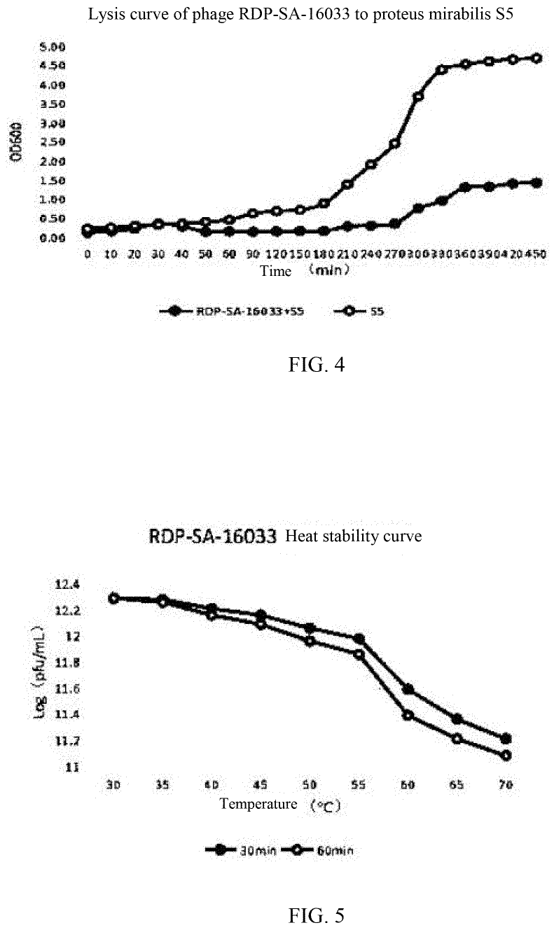

A phage stock solution was separately charged in EP tubes; the phages were respectively incubated under conditions such as 40° C., 50° C., 60° C. and 70° C. for 30 min and 60 min; and the phages were diluted with normal saline by 10 times, a double plate was laid, and titers of the phages were determined. The results are shown in FIG. 5.

Referring to FIG. 5, with temperature rise and extension of time, activity of the phage is decreased to a certain extent (respectively decreased by 1.1 and 1.2 order of magnitudes), which indicates that the RDP-SA-16033 has certain tolerance to temperature.

### Embodiment 10 Study on Genetic Stability Experiment

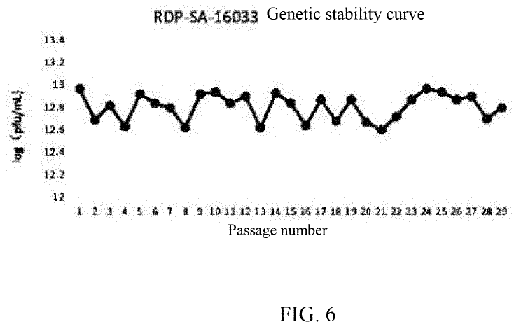

The RDP-SA-16033 was subjected to continuous passage for 29 times; equivalent RDP-SA-16033 and S5 were added into mediums per generation, wherein culture time of each generation is 6 h; and a double plate was laid, and the titer of the phage was determined. The results are shown in FIG. 6.

Referring to FIG. 6, after the RDP-SA-16033 is subjected to continuous passage for 29 times, the titer of the phage is stabilized at 10¹² pfu/mL or more, which indicates that the phage has excellent genetic stability ad is suitable for industrial production.

### Embodiment 11 Lysis Assay of Phage to Host

Under sterile conditions, 1 mL of sample and 1 mL of host bacteria solution (1×10⁵ CFU/mL) were respectively taken, incubated at 37° C. for 15 minutes, and diluted with normal saline to reach 10¹ to 10⁻³ gradients after uniform mixing; 100 μL of the mixture of each gradient was coated on an LB agar plate and cultured at 37° C. for 24 hours, and each gradient was repeated twice; meanwhile, 1 mL of normal saline and 1 mL of host bacteria solution (1×10⁵ CFU/mL) were taken as blank control, and the above steps were repeated; and a plate with 30-300 colonies was selected for counting. The assay was repeated for 3 times, and the mean value was taken. The lysis rate of the phage is equal to (1−the number of colonies in the treatment group/the number of colonies in the control group)×100%.

Through calculation, the lysis rate of the RDP-SA-16033 is up to 98%, has an excellent lysis effect on the host, and is suitable for application in the breeding process.

### Embodiment 12 RTD Experiment

A plate was zoned; a drop of host bacteria (about 100 μL) was dropped into the zone center and aired; and a drop of phage liquid of different degrees of dilution was dropped onto spots of the host bacteria, aired and cultured at 37° C. for 16-24 h. According to growth conditions of the host bacteria, all host bacteria that cannot continuously grow are called a CL concentration (the CL concentration is a concentration capable of completely lysing host bacteria in an environment, and may be used for guiding a dilution solution in production practice). The study shows that, the RDP-SA-16033 still has an excellent lysis effect on the host bacteria after diluted by 10⁸ times.

### Embodiment 13 Blood Entry Experiment of Phage

After oral administration of a broiler for 6 h, phage detection is performed by a PCR method. Results show that, the phage may be detected in heart, liver, lung, kidney, thymus gland and serum, which indicates that the phage may enter blood through oral administration and reach the heart, liver, lung, kidney and thymus gland by virtue of blood circulation.

The phage in the present invention has high titer up to 10¹² pfu/mL or more, and has excellent tolerance on the temperature and pH value. After the phage is subjected to continuous passage for 29 times, the titer of the phage is still maintained at above 10¹² pfu/mL. The phage has excellent genetic nature, and still has an excellent lysis effect on the host after diluted by 10⁸ times. After feeding, the phage may be detected in the heart, liver, lung, kidney, thymus gland and serum. Further, by virtue of animal experiments, the phage has an excellent effect of treating Proteus mirabilis diseases of chickens, and does not cause any adverse reaction after use. By virtue of complete genome sequencing, it is discovered that no lysogenic gene or virulence gene exists in the phage genes, and the safety of the phage is further verified.

It should be noted that the specific embodiments are only representative examples of the present invention. Apparently, the technical solutions of the present invention are not limited to the above embodiments, and may have many variations. The technical solutions obtained by those ordinary skilled in the art according to the disclosure of the present invention or the written description of the document without objection shall be regarded as the protection scope of the present invention.

1. A Proteus mirabilis phage RDP-SA-16033, wherein a host of the Proteus mirabilis phage RDP-SA-16033 is Proteus mirabilis S5; the phage may form a plaque having a diameter of 4-6 mm on a double-layer plate; when observed through an electron microscope, the phage has a head in polyhedral cubic symmetry, is coated by nucleic acid, has a diameter of about 70 nm, has a tail of about 150 nm long, has a tail sheath, has a neck connected to the head and the tail, and belongs to tailed virales myovirus division; and a collection number of the phage is CGMCC No. 18197.

2. An industrial production method of the phage RDP-SA-16033 of claim 1, comprising the following steps:
(1) seed preparation, comprising host seed preparation and phage seed preparation, wherein,
A, the host seed preparation: inoculating a single colony to a 5 mL of LB fluid medium in a sterile environment, and performing culture at 37° C. and 200 rpm for 4-6 h; inoculating the cultured host bacteria to a 100 mL of LB fluid medium at an inoculum size of 5%, and performing culture at 37° C. and 200 rpm for 4-6 h; inoculating the host bacteria to a 1000 mL of LB fluid medium at an inoculum size of 5°/o, and performing culture at 37° C. and 150 rpm for 4-6 h; and placing the cultured host bacteria seeds in a refrigerator at 4° C. for later use;
B, the phage seed preparation: properly diluting preserved phage seeds; placing 200 μL of the phage seeds and 200 μL of the host bacteria in an upper medium; pouring the medium into a double-layer plate; performing culture at 37° C. for 12-16 h; picking a plaque under a sterile condition, and placing the plaque in 1 mL of normal saline at 12000 rpm for 10 min; respectively inoculating the liquor and the host to a 5 mL of LB medium according to a ratio of 2%, and performing culture at 37° C. and 200 rpm for 4-8 h; simultaneously inoculating the cultured phage and the host to a 100 mL of LB fluid medium at an inoculum size of 2%, and performing culture at 37° C. and 200 rpm for 4-8 h; simultaneously inoculating the cultured phage and the host to a 1000 mL of LB fluid medium at an inoculum size of 2%, and performing culture at 37° C. and 200 rpm for 4-8 h; and placing the cultured phage seeds in the refrigerator at 4° C. for later use;
(2) phage proliferation: inoculating 3% of the host bacteria in a seed tank; after culturing for 4-6 h, controlling OD600 to 0.6-0.8; inoculating the phage seeds at an inoculum size of 3% and performing culture for 6-8 h until dissolved oxygen bubbles and the pH tends to be stable;
inoculating the prepared host bacteria in a seed fermenter according to a ratio of 3%; after culturing for 3-4 h, controlling the OD600 value to 0.6-0.8; inoculating the host bacteria in the fermenter by virtue of a seed transfer tube at an inoculum size of 5%; culturing the host in the seed tank for 4-6 h; inoculating the phage at an inoculum size of 5% when the OD value is 0.6-0.8, and performing culture for 6-8 h; and ending proliferation when the dissolved oxygen bubbles and the pH tends to be stable;
(3) phage after-treatment process:
A, centrifugation: centrifuging the completely proliferated phage by a tube centrifuge at a rate of 14000 rpm and flow velocity of less than or equal to 30 L/H, and removing non-lysed host and bacteria debris;
B, performing concentration by a spiral-wound membrane so as to increase a titer of the phage, wherein a specification of the spiral-wound membrane is 50 kd;
C, performing filtration by a 500 nm ceramic membrane, and removing partial bacteria debris and residual host;
D, filtration sterilization: adopting three-stage filtration: sterilizing by 0.45 μm polypropylene, 0.2 μm double-layer polypropylene and 0.22 μm polyether sulfone;
(4) low-temperature spray drying of the phage:
adding a carrier into the filtered and sterilized phage liquid, and uniformly stirring; performing low-temperature spray drying at a drying temperature of 60° C. and a feed rate of 8 L/H, thereby obtaining phage powder;

3. The industrial production method according to claim 2, wherein in the step (2), parameters of the seed tank are as follows: 200-300 rpm, 37° C., ventilating 1: (0.6-0.8) vvm; parameters of the fermenter are as follows: 120-150 rpm, 37° C., ventilating 1: (0.6-0.8) vvm.

4. The industrial production method according to claim 2, wherein in the step (2), the medium comprises the following components: 2% of yeast extract powder, 0.5% of glycerin, 0.5% of sodium chloride, 0.1% of monopotassium phosphate, 0.1% of dipotassium phosphate, 20 ppm of calcium chloride, 20 ppm of magnesium chloride and the balance of water.

5. The industrial production method according to claim 2, wherein in the step (4), the carrier comprises the following components in percentage by mass: 3% of soluble starch, 3% of polyvinylpyrrolidone, 3% of lactose, 2% of trehalose, 0.2% of disodium hydrogen phosphate, 0.3% of sodium dihydrogen phosphate, 0.1% of vitamin C and 0.5% of modified chitosan.
